(19) United States
(12) Patent Application Publication
(10) Pub. No.: US 2011/0230717 A1
(43) Pub. Date: Sep. 22, 2011

(54) Flexible endoscope external joint

(75) Inventors: Gregory S. Konstorum, Stamford, CT (US); Christopher A. Cook, New York, NY (US); Kazutaka Matsumoto, Riverside, CT (US)

(73) Assignee: Gyrus ACMI, Inc.

(21) Appl. No.: 12/661,504

(22) Filed: Mar. 18, 2010

(51) Int. Cl. A61B 1/005; B23P 17/04; H01R 43/00; B32B 37/06

(52) U.S. Cl. 600/140; 29/428; 29/592.1; 156/86

(57) ABSTRACT

An endoscope including a deflection section, an objective head, a deflection section cover and a sleeve. The deflection section comprises a flexible frame. The objective head is connected to a front end of the deflection section. The deflection section cover extends over the deflection section and extends over a portion of the objective head. The sleeve is located on an exterior side of the objective head and an exterior side of the deflection section cover at a joint between the objective head and the deflection section cover. The sleeve compresses the deflection section cover against the objective head. Another compression sleeve can be located on an exterior side of a shaft cover and the deflection section cover at a second joint, wherein the second sleeve compresses the shaft cover and the deflection section cover against a frame of a shaft of the endoscope.

### BACKGROUND OF THE INVENTION

1. Field of the Invention

The invention relates to an endoscope and, more particularly, to an external joint on an endoscope.

2. Brief Description of Prior Developments

Flexible endoscopes are known, such as described in U.S. Patent Application Publication No. 2003/0023142 A1. In the past, thread has been wrapped around adjacent components and covered with adhesive at exterior joints between a deflection section cover and the objective head, and between the deflection section cover and the shaft cover. However, this technique at exterior joints is labor intensive. In addition, this technique at exterior joints is not necessarily consistent. There is a desire to provide a less labor intensive and more consistent exterior joint formation on an endoscope.

### SUMMARY

The following summary is merely intended to be exemplary. The summary is not intended to limit the scope of the claimed invention.

In accordance with one aspect of the invention, an endoscope is provide including a deflection section, an objective head, a deflection section cover and a sleeve. The deflection section comprises a flexible frame. The objective head is connected to a front end of the deflection section. The deflection section cover extends over the deflection section and extends over a portion of the objective head. The sleeve is located on an exterior side of the objective head and an exterior side of the deflection section cover at a joint between the objective head and the deflection section cover. The sleeve compresses the deflection section cover against the objective head.

In accordance with another aspect of the invention, an endoscope is provide comprising a shaft, a deflection section, a deflection section cover, and a compression sleeve. The shaft comprises a frame and a shaft cover extending over the frame. The deflection section is connected to a front end of the shaft, wherein the deflection section comprises a flexible frame. The deflection section cover extends over the deflection section and extends over a portion of the frame of the shaft. The compression sleeve is located on an exterior side of the shaft cover and the deflection section cover at a joint, wherein the sleeve compresses the shaft cover and the deflection section cover against the frame of the shaft.

In accordance with another aspect of the invention, a method is provided comprising providing a deflection section cover on a flexible frame of an endoscope deflection section, wherein an end of the deflection section cover extends onto a first member of an endoscope, wherein the first member is an objective head of the endoscope or a portion of a frame of a shaft of the endoscope; and connecting a sleeve around an exterior side of the deflection section cover at an exterior side of the endoscope, wherein the sleeve compresses the end of the deflection section cover against the first member, and wherein the sleeve also compresses against the first member or a second member of the endoscope at a joint of the end of the deflection section cover to the first or second members.

### BRIEF DESCRIPTION OF THE DRAWINGS

The foregoing aspects and other features of the invention are explained in the following description, taken in connection with the accompanying drawings, wherein:

FIG. 1 is an elevational side view of an endoscope comprising features of the invention;

FIG. 2 is a schematic cross sectional view of a front end of the endoscope shown in FIG. 1;

FIG. 3 is a partial enlarged view of the rear joint of the deflection section cover of the endoscope shown in FIG. 2;

FIG. 4 is a partial enlarged view of the front joint of the deflection section cover of the endoscope shown in FIG. 2;

FIG. 5 is a perspective view of the sleeve used in the front and rear joints shown in FIGS. 3 and 4;

FIG. 6 is a perspective view of an alternate embodiment of the sleeve shown in FIG. 5;

FIG. 7 is a schematic cross sectional view similar to FIG. 4 showing a connection of the sleeve shown in FIG. 6 to the endoscope as an alternate embodiment; and

FIG. 8 is a schematic cross sectional view similar to FIG. 3 showing a connection of the sleeve shown in FIG. 6 to the endoscope as an alternate embodiment.

### DETAILED DESCRIPTION OF EMBODIMENTS

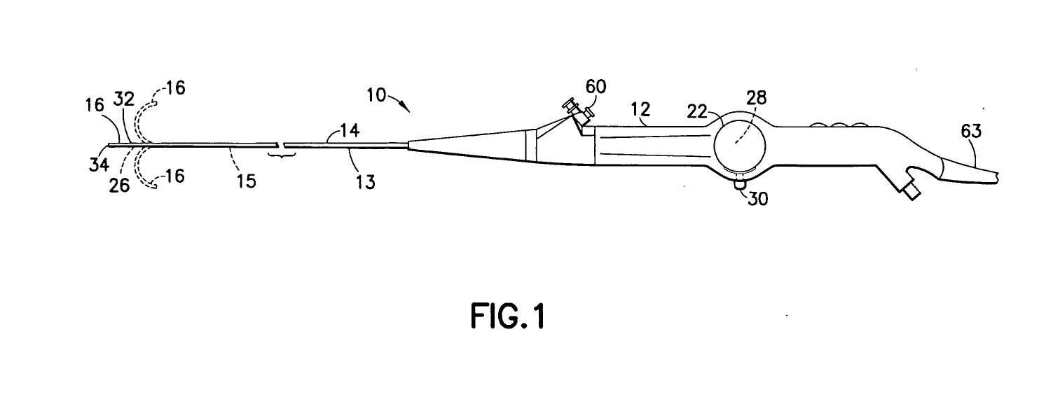

Referring to FIG. 1, there is shown a side view of an endoscope 10 incorporating features of the invention. Although the invention will be described with reference to the example embodiments shown in the drawings, it should be understood that the invention can be embodied in many alternate forms of embodiments. In addition, any suitable size, shape or type of elements or materials could be used.

The endoscope 10 is a ureteroscope. However, in alternate embodiments the endoscope could be any suitable type of endoscope. The endoscope 10 generally comprises a handle or control 12 and a flexible or semi-flexible or rigid shaft 14 connected to the handle 12. A deflection section (or steering section) 16 is located at a distal end of the shaft 14. The deflection section could include a passive deflection section and an active deflection section. A control system 22 to control the deflection section extends from the handle 12 to the deflection section. The control system 22 can comprise, for example, a pair of control wires, two wire sheaths, and an actuator 28. One end of the wires are connected to the actuator 28 and an opposite second end of the wires are connected to the distal end of the deflection section.

In the preferred embodiment, the handle 12 has a user operated slide or lever 30. The lever 30 is connected to the actuator 28. The actuator 28 is adapted to pull and release the two wires of the control system 22. When the lever 30 is moved by the user, the actuator is moved. The actuator 28 may be a drum or pulley, for example, rotatably connected to the handle 12 to pull one wire while releasing the other. In an alternate embodiment, the actuator may be any suitable type of device, such as a rocker arm adapted to pull and release the wires of the control system 22. In another alternate embodiment, where the control system may have two or more pairs of control wires, the handle can have additional actuators and corresponding controls to drive the additional pairs of control wires to bend the deflection section in different plane(s). In still other alternate embodiments, the handle may have knobs with rack and pinion mechanisms or other suitable user operated controls for the control system.

The shaft 14 is cantilevered from the handle 12. The shaft 14 and the deflection section 16 include the control wires of the control system 22, a fiber optical image bundle or video sensor electrical cable, a working channel, and a fiber optical illumination bundle or electrical wires to illumination LEDs or lights at the objective head 34. A port 60 for inserting accessory instruments (not shown) into the working channel is located on the handle 12. The handle 12 also has an electrical cable 63 for connection to another device, such as a video monitor for example. In an alternate embodiment, instead of the cable 63, the endoscope could have an eyepiece. In alternate embodiments, the flexible shaft may house different systems within.

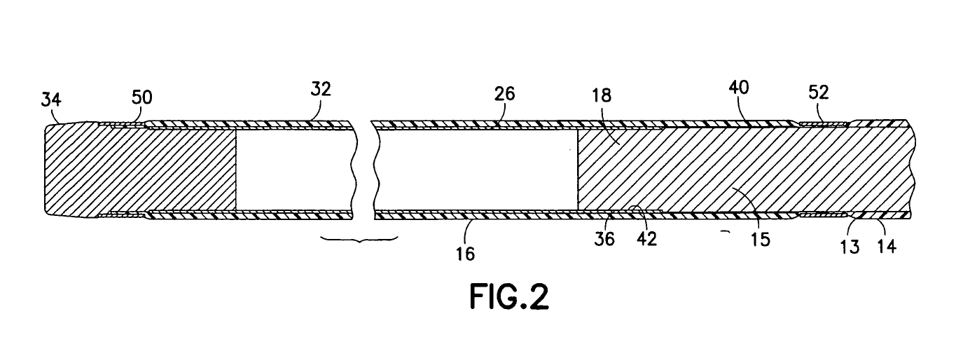

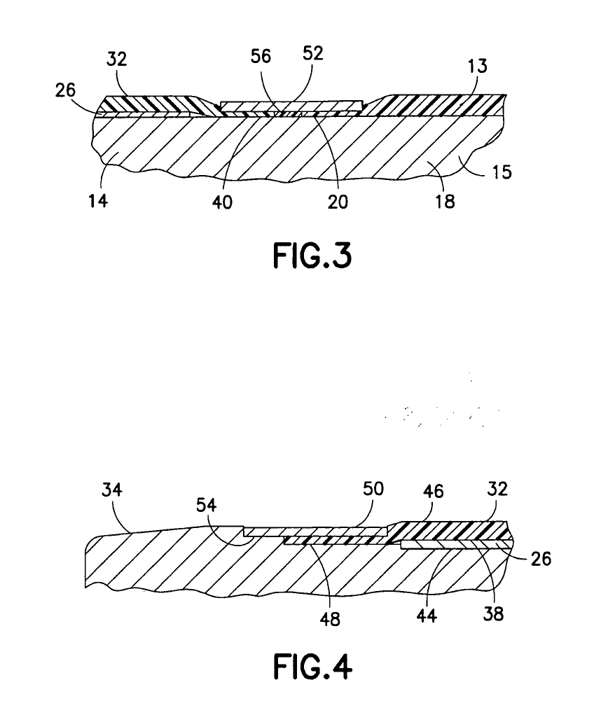

Referring also to FIG. 2, a schematic cross sectional view of the front end of the endoscope 10 is shown. The shaft 14 includes a frame 15 and a shaft cover 13. The frame 15 could be rigid or flexible, such as comprised of interconnected rings or a flexible tube for example. The shaft cover 13 preferably comprises a tube of plastic or polymer material. As seen in FIG. 3, the front end 18 of the frame 15 of the shaft 14 extends past the front end 20 of the shaft cover 13.

As seen in FIG. 2, the deflection section or steering/articulation section 16 generally comprises a frame 26 and a deflection section cover 32. The frame 26 is flexible, such as rings pivotably connected to each other in series or a tube of superelastic alloy, for example. The rear end 36 of the frame 26 is connected to the front end 18 of the shaft frame 15. In this embodiment the front end 18 of the frame 15 has a recessed pocket 42 for receiving the rear end 36 of the frame 26. Referring also to FIG. 4, the front end 38 of the frame 26 is connected to the rear end of the objective head 34. In this embodiment the objective head 34 has a recessed pocket 44 which receives the front end 38 of the frame 26.

The deflection section cover 32 has a general tube shape and is comprised of a flexible plastic or polymer material. The cover 32 surrounds the frame 26. A rear end 40 (see FIG. 3) of the cover 32 extends past the rear of the end 36 of the frame 26 and onto the frame 15. A front end 46 of the cover 32 extends past the front of the end 38 of the frame 26 and onto the objective head 34. In this example embodiment the objective head 34 has a recessed pocket 48 which receives the front end 46 of the cover 32.

Referring also to FIG. 5, the endoscope 10 further comprises two compression sleeves 50, 52. In this example embodiment the two sleeves 50, 52 are identical. However, in alternate embodiments the two sleeves could be different. In addition, more or less than two compression sleeves could be provided. The sleeves have a general loop or continuous ring shape. In this example embodiment each sleeve 50, 52 is comprised of a shape memory alloy which is a temperature shrinkable material which shrinks at a predetermined temperature. The material could be Tinel or Nitinol for example. The rings could be comprised of any one of a number of several different alloys, each formulated and processed to achieve specific functionality, such as Alloy H for example. This alloy has a Heat-To-Recover (H-T-R) characteristic that makes it suitable for most fastening and sealing applications.

A H-T-R ring offer new ways to join and seal cylindrical components. These metal rings can be supplied in an expanded condition, and subsequently shrink about 4.5 percent in diameter when heated. Once shrunk, the rings can apply a uniform gripping pressure that is seamless, powerful and consistent. The gripping force can be set between 220 N (50 lb) and 130 kN (30,000 lb) by choice of the ring dimensions for example.

H-T-R rings generally begin to shrink when heated above 45° C. (113° F.). Full clamping force can be achieved by heating to 165° C. (330° F.) for example. To insure full heating, H-T-R rings can be marked with temperature indicating paint that changes color at 165° C. for example. H-T-R rings can maintain their tight grip over an operating range of about −65° C. (−85° F.) to +300° C. (+572° F.). If the rings are cooled with liquid nitrogen, they can relax their grip on cooling below −120° C., allowing disassembly or repositioning.

The rings can produce highly reliable joints because the shrinkage and clamping pressure generated are intrinsic properties of the ring material. The invention can provide a unique product that enables efficient designs. Benefits over conventional techniques, such as crimping, welding, adhesives, elastic assembly and threaded fasteners, include:

operator insensitive assembly;

low installation temperature;

joins and seals dissimilar materials;

simple verification of correct installation;

repeatability;

seamless 360° clamping pressure;

immunity to vibration, shock, and thermal cycling;

chemical resistance; and

rotary balance.

For assembly, the ring can be positioned on the components and heat can be applied until the thermochromic paint changes color. However, thermochromic paint does not need to be used. The heat may be applied by any suitable means such as an oven, a hot air gun, infrared, direct electric resistance heating, etc.

The rings may be removed by cutting with abrasive or carbide tools. Alternately, H-T-R rings will relax their grip if cooled below −120° C. (−185° F.) using liquid nitrogen for example. Rings will re-grip on warming to room temperature. However, the gripping force is sensitive to changes in the substrate diameter.

As seen in FIGS. 2 and 4, the first sleeve 50 is located at the objective head 34. In this example embodiment the objective head 34 includes a recessed pocket 54 which receives the front end of the first sleeve 50. The rest of the front sleeve 50 compresses or clamps the front end 46 of the cover 32 against the objective head at the pocket 48. In one method of connecting the sleeve 50, the sleeve 50 is placed on the front end 46 of the cover 32 and over the pocket 54. The sleeve 50 is then heated. As the sleeve 50 is heated, it shrinks to subsequently clamp against the objective head in the pocket 54, and clamp the front end 46 of the cover 32 in the pocket 48. Thus, the sleeve 50 helps to form an external joint between the deflection section cover 32 and the objective head 34. This technique is much less labor intensive, and repeatably more consistent, than the conventional method of thread wrapped around the adjacent components and covered with adhesive. The two conventional components (thread and adhesive) are replaced with only one component; the sleeve 50. However, in an alternate embodiment, adhesive or epoxy could be used on the outside of the sleeve 50 and/or between the inside of the sleeve 50 and the objective head 34 and/or cover 32 such as when an additional holding force is desired (more than just the compression force provided by the shrunken sleeve) or when a cross-sectionally flat outer surface is desired (the adhesive or epoxy acting as a filler at the sleeve). An advantage of the invention, however, is that adhesive or epoxy does not need to be used on the outside of the sleeve 50 and/or between the inside of the sleeve 50 and the objective head 34 and/or cover 32. The clamping action of the sleeve 50 may be sufficient merely due to the nature of its shrinking and its very strong superelastic properties. In an alternate embodiment, however, the sleeve 50 might be comprised of a different material, such as stainless steel for example.

Referring also to FIG. 3, the second sleeve 52 is located at a second external joint between the deflection section cover 32 and shaft cover 13. In this embodiment the joint includes material 56 located in the joint which connects the second sleeve 52 to the frame 15 of the shaft 14. The material 56 is located between the two ends 20, 40. In one embodiment the material is a RTV (room-temperature vulcanizing) material or ring, which could be electrically insulating or electrically conductive. In another embodiment the material is an electrically conductive adhesive. Thus, the second sleeve 52 could be electrically connected to the frame 15 at the joint. The sleeve 52 could, thus, be used as an electrical component, such as a ground between a patient's body and the frame 15, or as an electrode.

Similar to the first sleeve 50, in one method of connecting the sleeve 52, the sleeve 52 is placed over the two ends 20, 40 of the covers 13, 32. The sleeve 52 is then heated. As the sleeve 52 is heated, it shrinks to subsequently clamp the two covers 13, 32 against the frame 15 of the shaft 14. Thus, the sleeve 52 helps to form an external joint between the deflection section cover 32 and the shaft cover 13. This technique is much less labor intensive, and repeatably more consistent, than the conventional method of thread wrapped around the adjacent components and covered with adhesive. The two conventional components (thread and adhesive) are replaced with only one component; the sleeve 52. However, in an alternate embodiment, adhesive or epoxy could be used on the outside of the sleeve 52 and/or between the inside of the sleeve 52 and the cover 13 and/or cover 32 such as when an additional holding force is desired (more than just the compression force provided by the shrunken sleeve) or when a cross-sectionally flat outer surface is desired (the adhesive or epoxy acting as a filler at the sleeve). An advantage of the invention, however, is that adhesive or epoxy does not need to be used on the outside of the sleeve 52 and/or between the inside of the sleeve 52 and the covers 13, 32. The clamping action of the sleeve 52 may be sufficient merely due to the nature of its shrinking and its very strong superelastic properties. In an alternate embodiment, however, the sleeve 52 might be comprised of a different material, such as steel for example. Rather than a method which comprises a temperature shrinking material, the sleeves 50 or 52 could be crimped or swaged onto the endoscope.

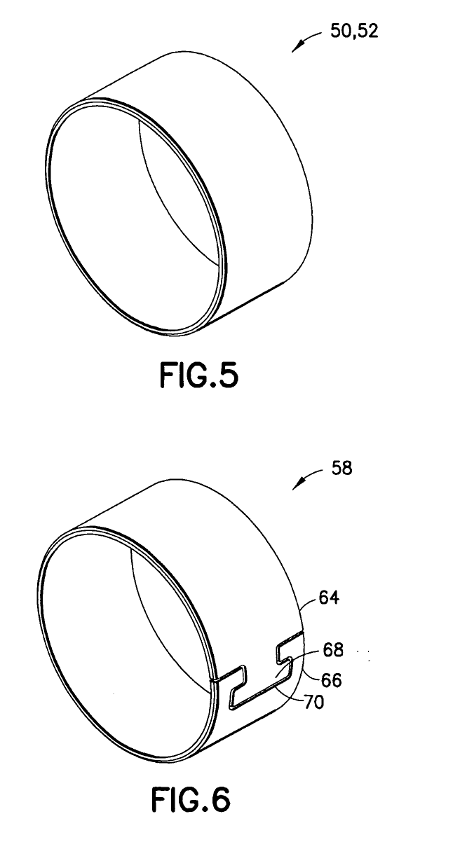

An example of another embodiment of the sleeve is shown in FIG. 6. In this embodiment the sleeve 58 is a self-locking sleeve. In particular, the sleeve 58 has two ends 64, 66 which are sized and shaped to mate with each other in an interlocking fashion. In the example embodiment shown, the first end 64 has a general T shaped projection 68, and the second end 66 has a general T shaped recess 70. However, in alternate embodiments other shapes could be provided. When the sleeve 58 is attached, it can compress the covers 13, 32 similar to the first embodiment described above, but without the need to use properties of a temperature shrinking material.

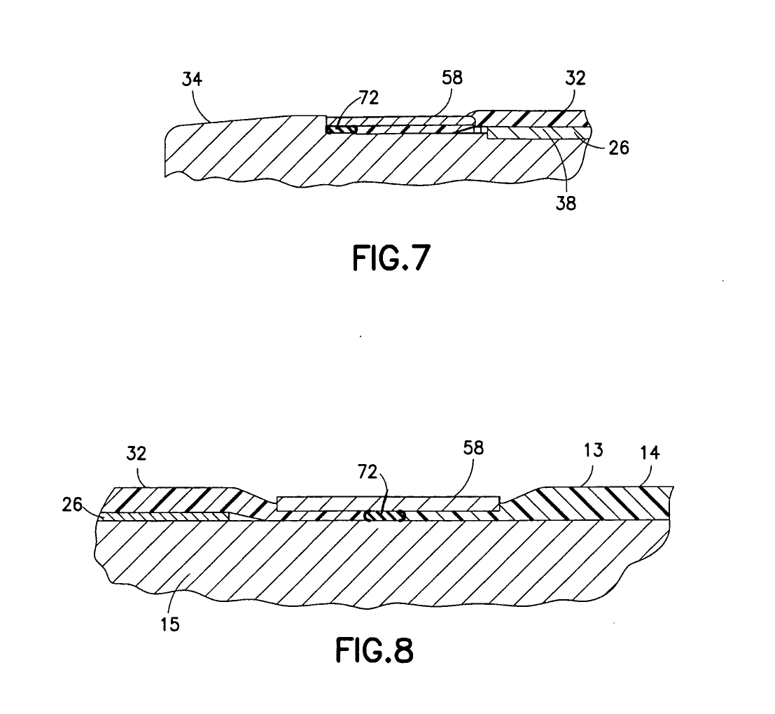

Referring also to FIG. 7, an example of use of the sleeve 58 at the objective head 34 is shown. The first joint at the objective head is substantially identical to the first embodiment except that that an electrically conductive O-ring 72 is provided to insure electrical connection of the sleeve 58 to the frame of the objective head.

Referring also to FIG. 8, an example of use of the sleeve 58 at the shaft 14 is shown. The second joint at the shaft 14 is substantially identical to the first embodiment except that an electrically conductive O-ring is provided rather than material 56 to insure electrical connection of the sleeve 58 to the frame 14 of the shaft 14.

With the invention an endoscope can be provided comprising a deflection section 16 comprising a flexible frame 26; an objective head 34 connected to a front end of the deflection section; a deflection section cover 32 extending over the deflection section frame 26 and extending over a portion of the objective head 34; and a sleeve 50 or 58 located on an exterior side of the objective head and an exterior side of the deflection section cover at a joint between the objective head and the deflection section cover, wherein the sleeve compresses the deflection section cover against the objective head. In one example embodiment the sleeve comprises a shape memory alloy. In one example embodiment the sleeve comprises a temperature shrinkable material which shrinks as the material is heated. In an alternate embodiment, the temperature shrinkable ring, if comprised of a suitable material, could be heated to expand, placed around the components, and then cooled towards room temperature in order to shrink. In one example embodiment the sleeve comprises a continuous loop ring shape. In one example embodiment the sleeve comprises a general ring shape with a joint having interlocked ends. In one example embodiment the sleeve comprises a one-piece metal member. In one example embodiment the sleeve could be swaged onto the objective head and deflection section cover. The endoscope can comprise a second sleeve which is located on an exterior side of a shaft cover of the endoscope and on an exterior side of the deflection section cover at a second joint, wherein the sleeve compresses the shaft cover and the deflection section cover against a frame of a shaft of the endoscope. The endoscope can comprise electrically conductive material located in the second joint which electrically connects the second sleeve to the frame of the shaft.

An endoscope can be provided comprising a shaft 14 comprising a frame 15 and a shaft cover 13 extending over the frame; a deflection section 16 connected to a front end of the shaft 14, wherein the deflection section comprises a flexible frame 26; a deflection section cover extending over the deflection section frame 26 and extending over a portion of the frame 15 of the shaft 14; and a compression sleeve 52 or 58 located on an exterior side of the shaft cover 32 and the deflection section cover 13 at a joint, wherein the sleeve compresses the shaft cover and the deflection section cover against the frame of the shaft.

A method of the invention can comprise providing a deflection section cover 32 on a flexible frame 26 of an endoscope deflection section 16, wherein an end of the deflection section cover extends onto a first member of an endoscope, wherein the first member is an objective head 34 of the endoscope or a portion of a frame 15 of a shaft 14 of the endoscope; and connecting a sleeve 50 or 52 or 58 around an exterior side of the deflection section cover 32 at an exterior side of the endoscope, wherein the sleeve compresses the end of the deflection section cover 32 against the first member, and wherein the sleeve also compresses against the first member or a second member of the endoscope at a joint of the end of the deflection section cover to the first or second members. The method of connecting the sleeve can comprise temperature shrinking the sleeve on the deflection section cover. The method of connecting the sleeve can comprise swaging the sleeve on the deflection section cover. The method can comprise the first member being the portion of the frame of the shaft, and the second member being a shaft cover extending over the frame of the shaft. The method can further comprise electrically connecting the sleeve to the frame of the shaft by electrically conductive material extending through the joint.

It should be understood that the foregoing description is only illustrative of the invention. Various alternatives and modifications can be devised by those skilled in the art without departing from the invention. For example, features recited in the various dependent claims could be combined with each other in any suitable combination(s). In addition, features from different embodiments described above could be selectively combined into a new embodiment. Accordingly, the invention is intended to embrace all such alternatives, modifications and variances which fall within the scope of the appended claims.

What is claimed is:

1. An endoscope comprising:
a deflection section comprising a flexible frame;
an objective head connected to a front end of the deflection section;
a deflection section cover extending over the deflection section and extending over a portion of the objective head; and
a sleeve located on an exterior side of the objective head and an exterior side of the deflection section cover at a joint between the objective head and the deflection section cover, wherein the sleeve compresses the deflection section cover against the objective head.

2. An endoscope as in claim 1 wherein the sleeve comprises a shape memory alloy.

3. An endoscope as in claim 1 wherein the sleeve comprises a temperature shrinkable material which shrinks as the material is heated.

4. An endoscope as in claim 1 wherein the sleeve comprises a continuous loop ring shape.

5. An endoscope as in claim 1 wherein the sleeve comprises a general ring shape with a joint having interlocked ends.

6. An endoscope as in claim 1 wherein the sleeve comprises a one-piece metal member.

7. An endoscope as in claim 1 wherein the sleeve is swaged onto the objective head and deflection section cover.

8. An endoscope as in claim 1 further comprising a second sleeve which is located on an exterior side of a shaft cover of the endoscope and on an exterior side of the deflection section cover at a second joint, wherein the sleeve compresses the shaft cover and the deflection section cover against a frame of a shaft of the endoscope.

9. An endoscope as in claim 9 further comprising electrically conductive material located in the second joint which electrically connects the second sleeve to the frame of the shaft.

10. An endoscope comprising:
a shaft comprising a frame and a shaft cover extending over the frame;
a deflection section connected to a front end of the shaft, wherein the deflection section comprises a flexible frame;
a deflection section cover extending over the deflection section and extending over a portion of the frame of the shaft; and
a compression sleeve located on an exterior side of the shaft cover and the deflection section cover at a joint, wherein the sleeve compresses the shaft cover and the deflection section cover against the frame of the shaft.

11. An endoscope as in claim 10 wherein the sleeve comprises a shape memory alloy.

12. An endoscope as in claim 10 wherein the sleeve comprises a Heat-To-Recover (H-T-R) temperature shrinkable material which shrinks as the material is heated to compress the shaft cover and the deflection section cover against the frame of the shaft.

13. An endoscope as in claim 10 wherein the sleeve comprises a continuous loop ring shape.

14. An endoscope as in claim 10 wherein the sleeve comprises a general ring shape with a joint having interlocked ends.

15. An endoscope as in claim 10 wherein the sleeve comprises a one-piece metal member.

16. An endoscope as in claim 10 wherein the sleeve is swaged onto the shaft cover and the deflection section cover.

17. A method comprising:
providing a deflection section cover on a flexible frame of an endoscope deflection section, wherein an end of the deflection section cover extends onto a first member of an endoscope, wherein the first member is an objective head of the endoscope or a portion of a frame of a shaft of the endoscope; and
connecting a sleeve around an exterior side of the deflection section cover at an exterior side of the endoscope, wherein the sleeve compresses the end of the deflection section cover against the first member, and wherein the sleeve also compresses against the first member or a second member of the endoscope at a joint of the end of the deflection section cover to the first or second members.

18. A method as in claim 17 wherein connecting the sleeve comprises temperature shrinking the sleeve on the deflection section cover.

19. A method as in claim 17 wherein connecting the sleeve comprises swaging the sleeve on the deflection section cover.

20. A method as in claim 17 wherein the first member is the portion of the frame of the shaft, and wherein the second member is a shaft cover extending over the frame of the shaft.

21. A method as in claim 20 further comprising electrically connecting the sleeve to the frame of the shaft by electrically conductive material extending through the joint.
